(19) United States
(12) Patent Application Publication
(10) Pub. No.: US 2006/0100182 A1
(43) Pub. Date: May 11, 2006

(54) Pharmaceutical compositions for the treatment of urinary incontinence

(76) Inventors: Caroline Rougaignon, Boulevard du Jardin Exotique (MC); Michel Lanquetin, La Trinite-Laghet (FR)

Correspondence Address: HEDMAN & COSTIGAN P.C., 1185 AVENUE OF THE AMERICAS, NEW YORK, NY, 10036, US

(21) Appl. No.: 10/524,131

(22) PCT Filed: Aug. 6, 2003
(86) PCT No.: PCT/FR03/02471

§ 371 Date: Sep. 13, 2005

(30) Foreign Application Priority Data
Aug. 7, 2002 (FR) 02 10058

(51) Int. Cl. A61K 31/56; A61K 31/195

(52) U.S. Cl. 514/171; 514/567

(57) ABSTRACT

Pharmaceutical compositions for the treatment of urinary incontinence with a cholinergic and musulotropic substance, combined or not combined with a little resorbed estrogen agent, by local route, wherein the cholinergic and musculotropic substance is oxybutynin, or one of its salts, and the little resorbed estrogen agent is chosen from estriol, 16-epiestriol or estradiol in free, esterified and/or etherified form suited for vaginal or rectal administration and a method of treating urinary incontinence.

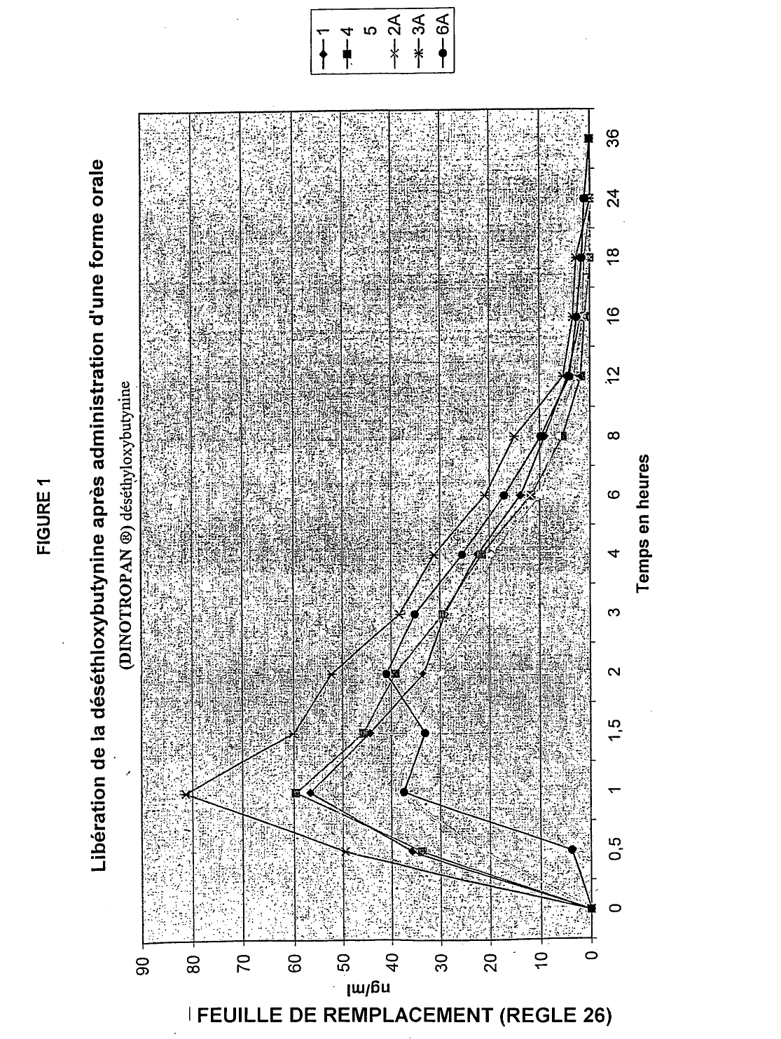

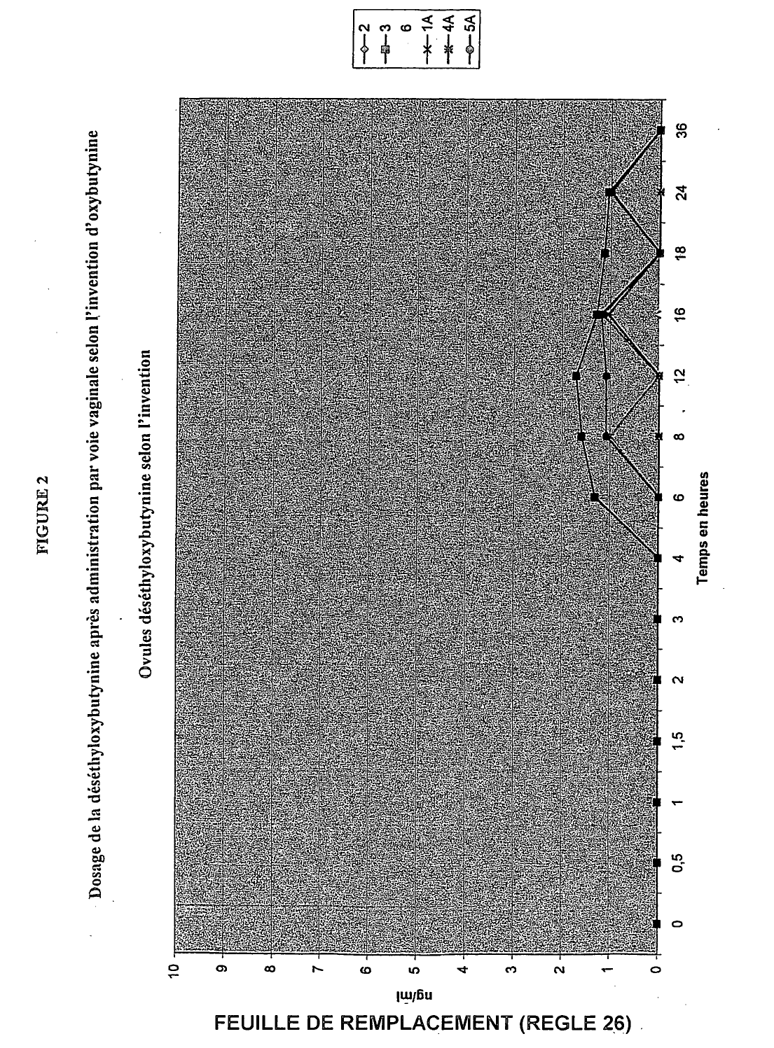

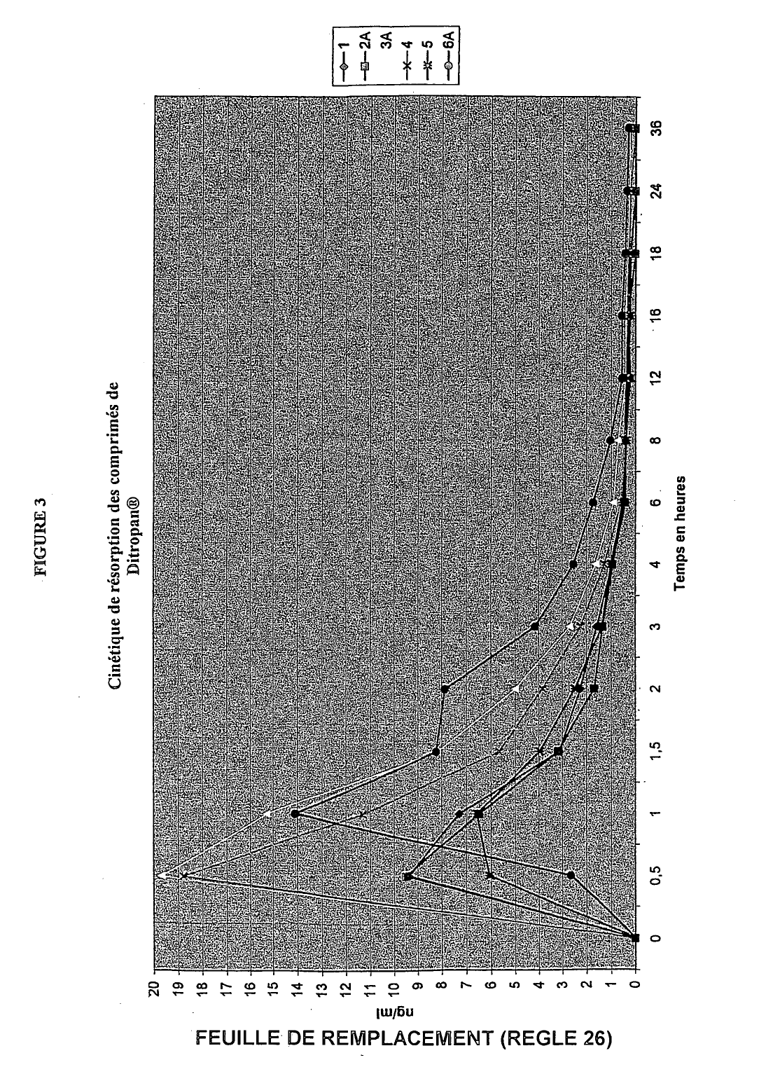

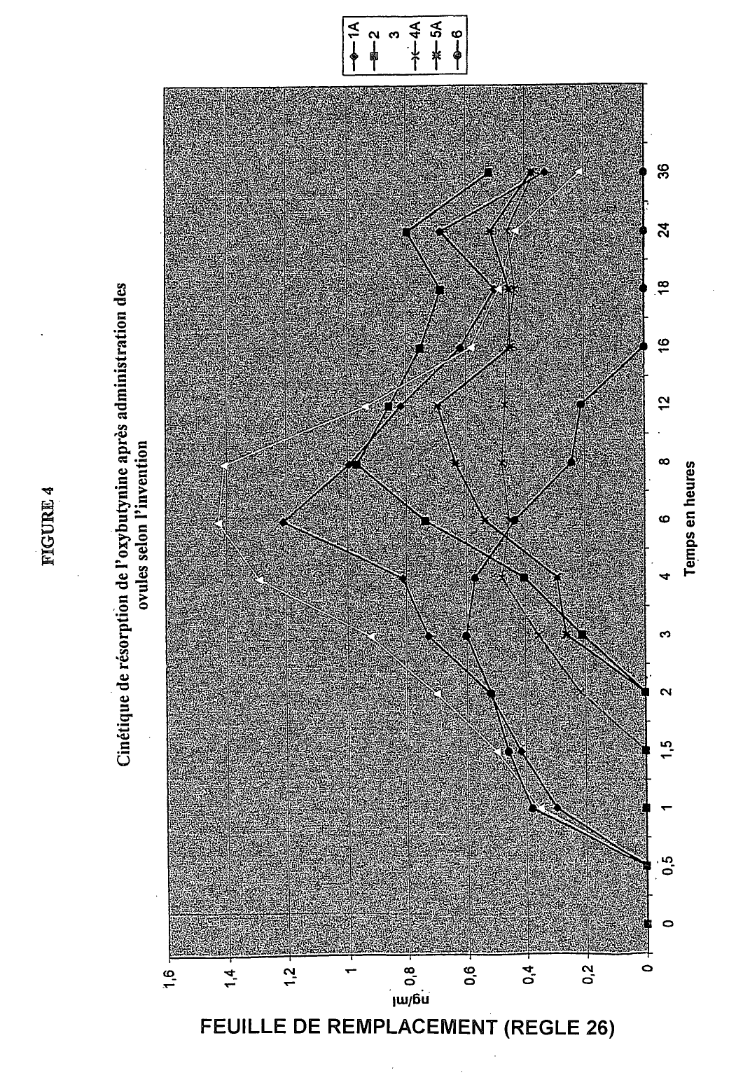

The present invention relates to the field of therapeutic chemistry and in particular to the field of pharmaceutical technology.

More particularly it has novel galenic forms intended for the treatment of urinary incontinence, in particular of urge incontinence and of detrusor instability in women.

It relates specifically to novel pharmaceutical compositions intended for the treatment of urinary incontinence containing a cholinergic and musculotropic substance, combined or not combined with a moderated estrogen agent which is little resorbed by local route, characterized in that the cholinergic substance is oxybutynin, in that the little resorbed estrogen agent is an estrogenic derivative chosen from estriol, 16-epiestriol, estradiol and their esterified and/or etherified derivatives and in that the administration is carried out in one of the forms which is suitable for the vaginal route or the rectal route.

In a combination of two active ingredients with very different chemical make-up, there still exists the problem of being able to achieve a release of the active ingredients which is balanced, while reducing the speed of absorption of one of the active ingredients and/or adapting the diffusion of the active ingredient which can be most quickly diffused to that of the active ingredient which is the least diffusable.

However, this technical principle is difficult to put into practice and it is observed, particularly in a general route, that in a mixture of active ingredients, one of the active ingredients is resorbed more quickly than the other or that the little resorbed active ingredient has an adverse effect on the resorption of the other active ingredient.

This is the reason why the problem of simultaneous resorption of several active ingredients is often a difficult problem to solve.

The problem with treating urinary incontinence by the administration of oxybutynin resides in the fact that this is an active ingredient which is very quickly resorbed by digestive route, and that its absorption is irregular and requires the addition of another active ingredient, in particular one of those which slows down resorption.

It is known that urinary incontinence affects approximately 20% of adults, and principally women, and has a considerable psychosocial impact, because it is an affection which impacts on all activities of everyday life. More particularly it affects women.

There are two types of urinary incontinence in women:
1. Stress incontinence, characterized by accidental urine leakage, which occurs, no matter how full the bladder is, during stress, and ceases with it. It is caused by poor functioning of the vesical sphincter.
2. Mictional urge incontinence which manifests itself in urine leakage preceded or accompanied by urinary urgency. It is the result of instability of the detrusor (bladder smooth muscle), which contracts in an erratic and uncontrollable manner, during the bladder filling phase.

The prevalence of incontinence increases with age and, after fifty years, mixed incontinence combining the two types described above is often encountered.

After the menopause, the end of the secretion of estrogens by the ovaries, which in the long run leads to vulvovaginal atrophy, also affects the mucosa of the urethra and of the vesical trigone, which are rich in estrogen receptors. This results in a decrease in the thickness of the mucosa and changes in the chorion which reduce the flexibility of the urethra and aggravate urinary problems.

The treatment of incontinence consists of administering a smooth muscle relaxant agent, such as oxybutynin which acts directly on the site in distal position in relation to the cholinergic receptor. The normal dose in a drug treatment consists of repeated doses of oxybutynin from two to four times per day. This type of administration is difficult to carry out because the administration needs to be made to conform to the treatment plan and this is unfavourable from the cost point of view. Moreover, oxybutynin is adversely affected by light and needs to be protected from the air. These properties do not help the formulation of a medicament in a form of administration which allows oxybutynin to be administered which resorbs it at a controlled and known rate, per unit of time, to produce the planned therapy.

Given the above therapeutic problem, scientists have resorted to the techniques of medical distribution and pharmaceutical distribution belonging to the present invention and all consider that there is an urgent need for a form of administration which can deliver the invaluable medicament that is oxybutynin, in a dose with a controlled flow rate, to patients in clinical need of treatment for incontinence. There is also an urgent need for a novel form of administration and to be able to have means for implementing a therapeutic treatment method allowing the delivery of oxybutynin at a controlled rate without this medicament exhibiting problematic side effects.

Oxybutynin is the active ingredient of the DITROPAN® specialty. Chemically it is 4-(diethylamino)-2-butynyl acetate ∝-cyclohexyl ∝-hydroxyphenyl hydrochloride.

The molecule comprises an asymmetrical carbon atom. The compound as well as its deethylated metabolite have already been resolved into (R)- and (S)-oxybutynin or into (R)- or (S)-deethyloxybutynin (Sepracor EP914113). S-oxybutynin has also been used in the treatment of urinary incontinence.

Of course instead of oxybutynin hydrochloride, another therapeutically acceptable salt of oxybutynin can be used with the same effectiveness, chosen in particular from the group constituted by acetate, bitartrate, citrate, edetate, edisylate, estolate, esylate, fumarate, gluceptate, gluconate, glutamate, hydrobromide, hydroiodide, lactate, malate, maleate, mandelate, mesylate, methylnitrate, mucate, napsylate, nitrate, pamoate, pantothenate, phosphate, salicylate, stearate, succinate, tannate and tartrate. The choice is only influenced by factors of solubility or speed of resorption.

The international patent application WO 95/23593 constitutes the closest prior art. In this document a treatment for urinary incontinence in women, and in particular for menopausal women is proposed, with a combination containing a substrate for nitric oxide-synthase (NOS) or a nitric oxide donor (NOD) and a progestin, an estrogen and or a partial estrogen agonist.

The substrate for the nitric oxide synthase is arginine. The donor of nitric oxide is sodium nitroprusside.

The NOD is administered by oral route or by transdermal route.

The estrogen used in this combination is preferably an estradiol ester such as estradiol valerate or estradiol benzoate, conjugated equine estrogens, 17β-estradiol or also estrone or estriol.

Also the U.S. Pat. No. 6,262,115 (Alza Corp.) describes a method for the treatment of incontinence which consists of administering by oral route a dose of oxybutynin presented in a sustained release form.

In fact, oxybutynin, a musculotropic medicament widely used for the treatment of urinary incontinence, has drawbacks which reside in the fact that it is a medicament which is quickly metabolized in the organism into its more toxic deethylated derivative having largely lost the musculotropic activity (Hughes, Xenobiotica (1992) 7 859-869). As a result, in order to maintain effective blood levels over a long period, it is necessary to produce a system with two tablets whose active ingredient is oxybutynin, a first tablet ensures a release of oxybutynin over a short period of time (for example less than 6 hours) and a second tablet releases oxybutynin over an extended period for example from 18 to 24 hours (see U.S. Pat. No. 6,148,359).

However, oxybutynin has problematic side effects of the cholinergic type, such as a dry mouth, accommodation disorders, constipation, tachycardia, vertigo, aggravation of psychiatric problems (Jonville A. P. et al. Thérapie 1992, 47 389-392). The increase in the doses of oxybutynin is therefore not an easy problem to resolve.

The subject of the present patent application consists of developping a galenic preparation with a long period of activity, which can be administered by vaginal route or by rectal route, of an active ingredient: oxybutynin, combined or not combined with an estrogenic derivative such as for example estriol.

The administration of oxybutinin by vaginal or rectal route has two advantages:
1. It allows a resorption of the active ingredient very close to the targeted organ, the vesical muscle.
2. It avoids the hepatic first-pass effect, which neccessarily occurs after an oral administration, it consequently reduces the quantity of active ingredient destroyed by the liver and it reduces, as a result, the doses which are therapeutically necessary.

The administration by vaginal and rectal route thus allows the use of smaller doses by increasing the local effect of the product, while reducing its cholinergic side effects caused by its effect on the organs other that the bladder.

The aim is also to develop a galenic formulation which ensures a sustained release which is as regular as possible of the active ingredient having systemic action during the nycthemeron, so as to compensate for the short half-life of oxybutynin and to avoid the need for repeated administrations during the day.

The subject of the invention is therefore to produce a galenic form which guarantees the sustained release of oxybutynin or one of its salts, so as to ensure a therapeutic coverage for at least twelve hours. This is the benefit of administration by vaginal route or by rectal route.

Another subject of the invention resides in the fact that there may be combined with oxybutynin or one of its salts an estrogen such as estriol whose trophic effects are added in a synergistic manner to those of oxybutynin, a direct muscle mediator.

Moreover, among the estrogenic derivatives, estriol is preferably used because this is a moderated estrogen, used in the treatment of local problems of menopause, and the general diffusion of which is not very large when it is administered by vaginal route.

A—Combination of Two Active Ingredients:

The applicant studied the oxybutynin which is currently marketed and presented in the form of tablets at a dose of 5 mg with a daily dosage for adults, of 2 to 4 tablets.

By oral route, this is absorbed quickly and, at the recommended doses, measurement of the plasmatic levels has shown maximums of 8 to 18 ng/ml at a maximum time (T′ max) comprised between 0.5 and 1.4 hours. The oxybutynin is also subjected to a large hepatic first-pass effect and a systemic bioavailability close to 6% results.

It has also been noted that estriol, a weak estrogen, known for its ability to locally improve urethral trophicity and to stimulate the alpha-adrenergic receptors, responsable for closing the neck of the bladder, was able to demonstrate its effect on urinary incontinence with a small vaginal diffusion in circulating systems.

The solution to the problem of the bioavailability of the invention consisted of inserting, either by vaginal or by rectal route for women, and by rectal route for men, oxybutynin, or one of its salts, in order to obtain a systemic sustained effect, with pharmacologically acceptable circulating levels, and also of combining, optionally, in women, an estrogenic substance which locally stimulates the ∝-adrenergic receptors responsable for closing the neck of the bladder, with a substance such as active oxybutynin by oral route, therefore with systemic effect; thus there is combination with an active ingredient known to improve a local trophicity and thus there is a beneficial effect on urinary incontinence.

B—Choice of Administration Route:

Depending on their administration method, as well as on their pharmacological behaviour, the active ingredients are oriented towards the vaginal route in women. The rectal route, being intended for men, does not contain an estrogen agent.

For oxybutynin a reduction in its rapid metabolization caused by the hepatic first-pass is carried out and thus its bioavailability is increased by seeking circulating levels lower than those encountered at the T′ max. They are more constant over time. Vaginal or rectal absorptions constitute opportunities to achieve these results.

As regards the estrogenic derivatives, given their low vaginal absorption and their essentially local pharmacological activity, the vaginal route is the most suitable in this case.

C—Oxybutynin by General Route:

Today oxybutynin, used in the treatment of urinary incontinence, is administered by oral route, the marketed products being tablets at a dose of 5 mg with immediate release.

This type of galenic, combined with the fact that the oxybutynin molecule has a short elimination half-life (2 hours), requires several daily administrations (up to 4) in order to be effective all day (statutory information DITROPAN®, Dictionaire Vidal 2001).

The result of this is the discomfort inherent in the requirement for several daily administrations, and each time these repetitions lead to a large plasmatic peak of the product (the oxybutynin undergoes a significant hepatic degradation by first-pass effect), which as a consequence causes well known undesirable effects, which are very bothersome for the patients (dictionnaire Vidal 2001 DITROPAN®). The patients, as a result, often abandon the treatment.

Currently, there is no sustained release form available from the medical profession. However, a certain number of these forms are being studied. They will undoubtedly be marketed soon.

This includes in particular DITROPAN® XL which reduces the number of incontinence episodes but which does not however allow the appearance of undesirable side effects to be avoided (Gleason et al., Urology, 54(3), 420-3, September 1999), which is explained by the systemic passage of the product equivalent to that of DITROPAN®.

Other administration routes are being developped which will allow a continuous diffusion of the product: for example the transdermal route (patent WO01/80796).

According to some authors, this route allows an effectiveness comparable to that of the oral route, but does not significantly eliminate the undesirable effects inherent in the systemic passage of oxybutynin which leads certain patients to abandon their treatment (Ho C., Issues Health. Technol., (24), 1-4, October 2001).

D—Oxybutynin by Local (Urogenital) Route:

The vesical administration of oxybutynin by means of a catheter is used for some patients.

This administration route allows a local effect of the product to be obtained, directly in the vesical muscle and as a result allows the intensity of the undesirable effects to be reduced (Lethoranta K. Scand. J. Urol. Nephrol., 36, 18-24, 2002/Ferrera P. et al., B. J. U. Int., 87(7), 674-8, May 2001/Distasi S. M. et al., J. Urol., 165(2), 491-8, February 2001).

The drawback of the vesical route resides in the fact that this administration route is only possible using an intra urethral catheter, which by definition limits the number of patients who can benefit from it.

The administration of the product in a neighbouring cavity, the vagina, must be envisaged. It can be carried out with different forms:

1—The Vaginal Ring

The vaginal ring is a device which is implanted in the vaginal cavity of the patient and which will release the oxybutynin in a continuous manner for 28 days (patent WO 01/70154) (Schroder A. et al., Urology, 56(6), 1063-7, December 2000).

The main drawback is the constant presence of the ring and all that this involves in terms of discomfort, asepsis or local reaction.

2—Vaginal or Rectal Suppository

In order to avoid this major drawback, the applicant has developped a vaginal or rectal suppository of oxybutynin, or one of its salts, combined or not combined with an estrogenic derivative, which can be administered easily and which is well tolerated, both at a local and systemic level.

The benefit of the combination and of the pharmaceutical form are explained in the following paragraphs.

The present invention therefore relates to the administration by vaginal or rectal route of a suppository containing oxybutynin, combined or not combined with an estrogen derived from estradiol or estriol. In fact, to harmonize all the hypotheses, the choice of a sustained release formulation appears to be the most appropriate.

The vaginal or rectal administration routes have not been much used for this type of medicament until now. The experiment has already allowed the production of an effective form of a salt of the active ingredient. This formulation allows, through a sustained period of contact with the mucosa, a more regular passage of oxybutynin to be obtained in the circulating systems and less metabolization, and, in the case of estriol, this is, moreover, directly resorbed on its action site by the vaginal route.

The formulation according to the invention, is composed of a combination of semisynthetic glycerides with suitable melting points, having different lipophilic characteristics, characterized by their hydroxyl value, these being known to modify the release profiles of certain active ingredients. Silica is added. The silica is used, for two very specific purposes:
a) to maintain the homogeneity of the suspension of the insoluble active ingredient in the fatty substance,
b) to give the formulation a bioadhesive character during the melting of the fatty substance in the vagina or the rectal cavity, through hydrogen bonds between the proteins of the vaginal or rectal mucus and the acid groups of the silicic acids.

The advantage of such a formulation resides in the fact of having, in the case of a combination, an active ingredient dissolved in the oil phase (estriol) and another maintained in suspension (oxybutynin hydrochloride).

Owing to their respective chemical structures, in the case of a combination, it is not expected that incompatibility between these two substances will be encountered. Verification was carried out during tests of stability of the form, under ICH conditions.

Methods of Study and Evaluation of the Formulation:

These are carried out “in vitro” by comparative dissolution (lyoavailability) studies.

The formulations selected “in vitro” were confirmed by “in vivo” studies, presented in the experimental part.

Research on the definitive doses was carried out once the first absorption kinetics of the active ingredients were produced.

The subject of the invention is therefore defined as the production of a pharmaceutical form allowing the administration which is best suited to the pathology of an incontinent patient, with reduced side effects.

According to a preferred embodiment, the active ingredients are oxybutynin in the form of a base, or a salt, with a therapeutically compatible mineral or organic acid, in racemic or optically active (epimers) form and, in the case of a combination, an estrogenic active ingredient derived from estradiol or from estriol, dissolved in an excipient or a fatty vehicle, suitable for administration by vaginal route and the other in suspension (oxybutynin hydrochloride).

Among the estradiol esters, there may be mentioned the acetates, butyrates, propionates, nicotinates, salicylates, cyclopentylpropionates, enanthates, hemisuccinate and cyclohexyl acetates. Among the estradiol ethers, the symmetrical diethers of the two alcohol or phenol functions can be mentioned such as for example 3, 17-dimethoxy-estradiol or of different ethers such as for example 3-propyloxy 17-methoxy estradiol or also mixed ether/ester structures such as 3-acetoxy, 17-methoxy estratriene or 3-propionyloxy 17-methoxy estratriene.

Among the derivatives of estriol, 3-methoxy 16,17-dinicotinoyloxy estratriene or 3,16-diacetoxy estra 1,3,5(10)-triene 17-one may be mentioned.

The solid semisynthetic glycerides are chosen from Witepsol® WS or WH19 and Suppocire® NA 16, NA I 50. They are used as fatty substances for the production of suppositories.

The choice is determined by the value of the melting point (generally as close as possible to 37° C.), the nature of the viscosity close to the melting point and their hydroxyl value.

Optimization of the release between the fatty materials, by varying of the proportions of one and the other, showed that the best fraction in terms of slow release of the active ingredient was a mixture in approximately equal proportions of Witepsol® WH 19 and of Suppocire® NAI 50.

Hydophilic agents of the PEG 4000 to 6000 type can be added to the fatty substances in order to affect the melting points of the semisynthetic glycerides and to modify the release profiles.

Among the suspension agents incorporated in the formulation, different qualities of silica will be noted such as for example AEROSIL® 200, AEROSIL® R992, the products COR84 and 300 from the Degussa company which are distinguished from each other by the lipophilic or hydrophilic character of each one.

The percentage of suspension agent can be comprised between 0 and 10% but preferably between 1.5% and 5% as a function of the desired release profile.

The formulation according to the invention also contains one or more gelling agents which improve the adhesion of the forms to the vaginal or rectal walls. The gelling agents according to the invention are cellulose derivatives and in particular alkylated or hydroxyalkylated cellulose derivatives. In this respect hydroxypropyl celluloses (HPC), (hydroxypropyl) methyl celluloses (HPMC), hydroxy ethyl methyl cellulose can be mentioned. They are present in quantities ranging from 5 to 20% of the formulation. Preferably HPMC such as those of the SM4000 or 6J-60-90 SM4000 type, as well as those known as 90 SH 100 000 are used.

The hydrogels formed from these gelling agents are quite sensitive to shearing and require a very complicated industrial application. The preferred products are those marketed under the Metolose trade mark (Shin-Etsu). As a carbomer gelling agent and more particularly polycarbophil which forms an “in situ” gel with aqueous liquids of the vaginal or anal region can also be used.

The dose of oxybutynin, or one of its salts, contained in the suppositories is comprised between 1 and 25 mg and more particularly between 5 and 15 mg of oxybutynin hydrochloride. In a combination, the dose of little resorbed moderated estrogen, is comprised between 0.01 and 5 mg. The dose of estriol or of its esters or ethers is comprised between 0.1 mg and 2 mg. It is preferably comprised between 0.2 mg and 1 mg per unit dose.

Another subject of the invention is the production of a formulation, the administration of which to the patient, by rectal or vaginal route, is easy.

Another subject of the invention resides in the fact that it is thus possible to reduce the administrable doses of oxybutynin during a nycthemeron.

Another subject of the invention is the development of a form of administration with a sustained period of activity, whose side effects linked to the presence of oxybutynin are reduced appreciably and even eliminated.

Pharmacokinetics Results:

The study of phamacokinetics with the compositions according to the invention compared to a specialized form of oxybutynin hydrochloride.

The clinical study was carried out by comparing the administration by vaginal route and the administration by oral route. It was carried out as a crossover study of six randomized patients, with a wash-out period of seven days between the two treatment periods. Each patient receives the two forms of medication, at a dose of 5 mg.

### RESULTS

The attached tables I and II bring together the results of the blood levels of oxybutynin metabolite as a function of time.

The subjects referenced 2A, 3A and 6A are those who started in period 2.

The blood levels of deethyloxybutynin are measured over a period of thirty-six hours. For the first eighteen hours, the blood levels of deethyloxybutynin are perceptible after administration of the commercial form of oxybutynin.

By contrast, by administering suppositories according to the invention, the blood levels of deethyloxybutynin are very low and it is the blood levels of oxybutynin which are really significant, as can be seen in tabler 1 and 2. They show the speed of absorption and of metabolization of the commercial product while the absorption with the suppositories according to the invention shows a maximum peak of oxybutynin which is much flatter and slightly delayed, with a resorption still perceptible after thirty-six hours, the maximum absorption being situated between six and eight hours, as is seen in tabler 3 and 4 illustrating these results.

It can therefore be seen that contrary to the administration by oral route of oxybutynin, which produces the N-deethylated metabolite, the administration by vaginal route or by rectal route leads to high and sustained blood levels of oxybutynin.

### EXAMPLES

The following examples illustrate the invention. They do not limit it in any way.

### EXAMPLE I

Gynecological capsule for vaginal administration
Unit formulation for a capsule

| | | |
|---|---|---|
| Oxybutynin hydrochloride | 5 | mg |
| Estriol | 1 | mg |
| Vaseline | 0.200 | g |
| Colloidal silica | 0.100 | g |
| Sorbitol sesquioleate | 0.05 | g |
| Perhydrosqualene qs. | | |

Coating: Gelatin, glycerol, preservatives qs. for a capsule with a weight of 1.85 g

### EXAMPLE II

Vaginal suppository
Unit formulation for a suppository

| | | |
|---|---|---|
| Oxybutynin hydrochloride | 5 | mg |
| Estriol | 0.5 | mg |
| Witepsol ® H35 | 1.4 | g |
| Witepsol ® H37 | 1.6 | g |
| qs. for a suppository weighing | 3.0055 | g |

### EXAMPLE III

Sustained release vaginal suppository
Unit formulation

| | | |
|---|---|---|
| Estradiol | 1.0 | mg |
| Oxybutynin hydrochloride | 7.5 | mg |
| Witepsol ® H19 | 1.5 | g |
| Witepsol ® H35 | 1.2 | g |
| Suppocire ® BM | 0.3 | g |
| Precirol ® | 0.3 | g |

Vaginal suppositories with an average weight of 3.3085 g are thus prepared.

### EXAMPLE IV

Compact bioadhesive gel for gynaecological use

| | | |
|---|---|---|
| Oxybutynin hydrochloride | 5.00 | mg |
| Estriol | 1.00 | mg |
| Polyethylene glycol 4000 | 1.00 | g |
| Transcutol ® | 5.00 | g |
| Polyvinyl carboxylic acid polymer | 1.000 | g |
| Preservative | 0.30 | mg |
| Triethanolamine qs. for pH 6.5 | | |
| Purified water qs. for 100 g | | |
| This gel is distributed in doses using a metering | 4 | g |
| pump equipped with a canula | | |

### EXAMPLE V

For a 2 g suppository (suppository 3)

| | | |
|---|---|---|
| Witepsol ® H19 | 0.965 | g |
| Suppocire ® NAI 50 | 0.965 g | |
| Aerosil ® 200 | 0.060 | g |
| Oxybutynin hydrochloride | 0.010 | g |
| Estriol | 1.5 | mg |

M.p. of the suppository 34.9° C.
Melting time 23 min
PA. active ingredient released in 6 H = 30%

### EXAMPLE VI

For a 2 g suppository (suppository 4)

| | |
|---|---|
| Witepsol ® H19 | 0.995 g |
| Suppocire ® | 0.995 g |
| Oxybutynin hydrochloride | 0.010 g |
| Estriol | 0.005 g |

M.p. of the suppository 34.5° C.
Melting time 19 min
PA. released in 6 H = 75%

### EXAMPLE VII

For a 2 g suppository (suppository 1)

| | |
|---|---|
| Witepsol ® H19 | 0.960 g |
| PEG 6000 | 0.010 g |
| Aerosil ® 300 | 0.060 g |
| Oxybutynin hydrochloride | 0.010 g |
| Estriol | 0.001 g |
| Suppocire ® NAI 50 | 0.959 g |

M.p. 36° C.
Melting time 38 min
PA. released in 6 H = 52%

### EXAMPLE VIII

For a 2 g suppository (suppository 2

| | |
|---|---|
| Witepsol ® H19 | 0.960 g |
| PEG 4000 | 0.010 g |
| Aerosil ® 200 | 0.060 g |
| Oxybutynin hydrochloride | 0.010 g |
| Estradiol methyl ether | 0.002 g |
| Suppocire ® NAI 50 | 0.958 g |

M.p. 35° C.
Melting time 30 min
PA. released in 6 H = 60%

### EXAMPLE IX

Soft elastic capsule

| | |
|---|---|
| Oxybutynin hydrochloride | 10 mg |
| Peanut oil | 100 mg |
| Polycarbophil | 60 mg |
| Envelope | |
| Gelatin, colorant | |

### EXAMPLE X

For a 2 g suppository

| | |
|---|---|
| Witepsol ® H19 | 0.950 g |
| Suppocire ® NAI 15 | 0.950 g |
| Colloidal silica (Aerosil ® 200) | 0.070 g |
| Carboxylic polyvinyl acid | 0.019 g |
| (Carbopol 1382) | |
| Estriol | 0.001 g |
| Oxybutynin hydrochloride | 0.010 g |

### EXAMPLE XI

For a 2 g suppository

| | |
|---|---|
| Witepsol ® S590 | 0.950 g |
| Suppocire ® NAI 50 | 0.950 g |
| H.P.M.C (Hydroxypropyl methylcellulose). | 0.078 g |
| Oxybutynin hydrochloride | 0.020 g |
| Estriol | 0.002 g |

### EXAMPLE XII

For a 2 g suppository

| | |
|---|---|
| Witepsol ® H19 | 0.960 g |
| Suppocire ® NAI 50 | 0.960 g |
| 3-propyloxy 17-methoxy estradiol | 0.002 g |
| Oxybutynin hydrochloride | 0.015 g |
| Polycarbophil in the form of the | 0.063 g |
| calcium salt (Goodrich) | |

### EXAMPLE XIII

For a 3 g vaginal suppository

| | |
|---|---|
| Witepsol ® H19 | 1.4525 g to 1.4325 g |
| Suppocire ® NAI 50 | 1.4525 g to 1.4325 g |
| Aerosil ® 200 | 0.0900 g to 0.1200 g |
| Oxybutynin hydrochloride | 0.0050 g to 0.0150 g |

### EXAMPLE XIV

For a 2 g rectal suppository

| | |
|---|---|
| Witepsol ® H19 | 0.935 g |
| Suppocire ® NAI 50 | 0.935 g |
| Aerosil ® 200 | 0.120 g |
| Oxybutynin hydrochloride | 0.010 g |

For a suppository completed at approximately 2 g.

TABLE I
Assay of deethyloxybutynin after administration of oxybutynin

| | | | | | | | | N^{o} of subjects with suppositories | | | | | |
|---|---|---|---|---|---|---|---|---|---|---|---|---|---|
| T′ in | N^{o} of subjects with Ditropan ® | | | | | | T′ in | according to the invention | | | | | |
| Hours | 1 | 4 | 5 | 2A | 3A | 6A | Hours | 2 | 3 | 6 | 1A | 4A | 5A |
| 0 | 0 | 0 | 0 | 0 | 0 | 0 | 0 | 0 | 0 | 0 | 0 | 0 | 0 |
| 0.5 | 35.8 | 33.8 | 21.6 | 46.5 | 49.5 | 3.67 | 0.5 | 0 | 0 | 0 | 0 | 0 | 0 |
| 1 | 56.7 | 59.6 | 38.5 | 52.7 | 81.3 | 37.4 | 1 | 0 | 0 | 0 | 0 | 0 | 0 |
| 1.5 | 44.3 | 45.6 | 36.2 | 39.8 | 60.1 | 33.1 | 1.5 | 0 | 0 | 0 | 0 | 0 | 0 |
| 2 | 33.3 | 39.1 | 30.0 | 32.6 | 52.3 | 40.9 | 2 | 0 | 0 | 0 | 0 | 0 | 0 |
| 3 | 29.0 | 29.4 | 22.3 | 25.0 | 38.2 | 35.1 | 3 | 0 | 0 | 0 | 0 | 0 | 0 |
| 4 | 22.3 | 21.5 | 19.7 | 18.7 | 31.2 | 25.4 | 4 | 0 | 0 | 0 | 0 | 0 | 0 |
| 6 | 13.8 | 11.7 | 10.2 | 11.9 | 20.9 | 17 | 6 | 0 | 1.31 | 0 | 0 | 0 | 0 |
| 8 | 9.00 | 5.32 | 6.49 | 7.12 | 15.0 | 9.78 | 8 | 0 | 1.59 | 0 | 1.01 | 0 | 1.08 |
| 12 | 4.27 | 1.71 | 3.32 | 2.63 | 5.19 | 3.86 | 12 | 0 | 1.71 | 0 | 0 | 0 | 1.08 |
| 16 | 1.65 | 1.01 | 1.64 | 1.31 | 3.24 | 2.5 | 16 | 0 | 1.28 | 0 | 1.16 | 1.05 | 1.19 |
| 18 | 1.16 | 0 | 1.23 | 0 | 2.58 | 1.53 | 18 | 0 | 1.13 | 0 | 0 | 0 | 0 |
| 24 | 0 | 0 | 0 | 0 | 0 | 1.07 | 24 | 1.02 | 1.05 | 0 | 0 | 0 | 1 |
| 36 | 0 | 0 | 0 | 0 | 0 | 0 | 36 | 0 | 0 | 0 | 0 | 0 | 0 |

1 = Subject 1 period 1
1A = Subject 1 period 2

TABLE II
Assay of oxybutynin in the blood after administration by vaginal route according to the invention or
by oral route (Ditropan ®)

| | | | |
|---|---|---|---|
| | N^{o} of subjects with suppositories | | |
| T′ in | according to the invention | T′ in | N^{o} of subjects with Ditropan ® |

| Hours | 1A | 2 | 3 | 4A | 5A | 6 | Hours | 1 | 2A | 3A | 4 | 5 | 6A |
|---|---|---|---|---|---|---|---|---|---|---|---|---|---|
| 0 | 0 | 0 | 0 | 0 | 0 | 0 | 0 | 0 | 0 | 0 | 0 | 0 | 0 |
| 0.5 | 0 | 0 | 0 | 0 | 0 | 0 | 0.5 | 9.38 | 9.5 | 19.7 | 18.8 | 6.07 | 2.65 |
| 1 | 0.299 | 0 | 0.354 | 0 | 0 | 0.38 | 1 | 7.31 | 6.48 | 15.3 | 11.3 | 6.58 | 14.1 |
| 1.5 | 0.418 | 0 | 0.501 | 0 | 0 | 0.461 | 1.5 | 3.13 | 3.19 | 8.17 | 5.66 | 3.97 | 8.24 |
| 2 | 0.524 | 0 | 0.701 | 0.218 | 0 | 0.518 | 2 | 2.26 | 1.7 | 5 | 3.86 | 2.49 | 7.88 |
| 3 | 0.727 | 0.21 | 0.925 | 0.356 | 0.263 | 0.602 | 3 | 1.6 | 1.35 | 2.7 | 2.24 | 1.37 | 4.15 |
| 4 | 0.812 | 0.403 | 1.29 | 0.48 | 0.293 | 0.571 | 4 | 0.963 | 0.945 | 1.64 | 1.28 | 1.02 | 2.54 |
| 6 | 1.21 | 0.736 | 1.43 | 0.454 | 0.539 | 0.434 | 6 | 0.55 | 0.428 | 0.931 | 0.613 | 0.501 | 1.75 |
| 8 | 0.996 | 0.97 | 1.41 | 0.48 | 0.637 | 0.243 | 8 | 0.446 | 0.413 | 0.721 | 0.436 | 0.376 | 1.05 |
| 12 | 0.818 | 0.858 | 0.941 | 0.469 | 0.694 | 0.21 | 12 | 0.378 | 0.205 | 0.39 | 0.295 | 0.306 | 0.555 |
| 16 | 0.619 | 0.751 | 0.583 | 0.46 | 0.448 | 0 | 16 | 0.295 | 0.31 | 0.408 | 0.259 | 0.216 | 0.561 |
| 18 | 0.502 | 0.683 | 0.489 | 0.433 | 0.453 | 0 | 18 | 0.298 | 0 | 0.322 | 0.254 | 0.21 | 0.439 |
| 24 | 0.685 | 0.795 | 0.431 | 0.46 | 0.517 | 0 | 24 | 0.253 | 0 | 0.259 | 0.208 | 0 | 0.361 |
| 36 | 0.331 | 0.521 | 0.215 | 0.367 | 0.377 | 0 | 36 | 0.205 | 0 | 0.23 | 0 | 0 | 0.3 |

1 = Subject 1 period 1
1A = Subject 1 period 2

1. A pharmaceutical composition for the treatment of urinary incontinence comprising oxybutynin as active ingredient, in combination or not with a moderated estrogen and a pharmaceutically acceptable excipient intended for vaginal or rectal administration.

2. A pharmaceutical composition of claim 1, wherein oxybutynin is selected from the group consisting of oxybutynin base, its addition salts with a mineral or organic acid and their epimers.

3. A pharmaceutical composition of claim 1, wherein the moderated estrogen is selected from the group consisting of estriol, estradiol and esters, ethers and mixed ethers of estriol and estradiol.

4. A pharmaceutical composition of claim 1, wherein it is selected from the form consisting of suppositories, vaginal capsules, rectal capsules and gels.

5. A pharmaceutical composition of claim 1 wherein it contains from 1 to 25 mg of oxybutynin or its salts.

6. A pharmaceutical composition of claim 5, wherein it contains from 5 to 15 mg of oxybutynin hydrochloride.

7. A pharmaceutical composition of claim 1 wherein it contains a dose of little resorbed moderated estrogen ranging from 0.01 to 5 mg per unit dose.

8. A pharmaceutical composition of claim 7, wherein the moderated estrogen is estriol at a dose of 0.1 to 2 mg.

9. A pharmaceutical composition of claim 7 wherein the unit dose of estriol ranges from 0.2 mg to 1 mg.

10. A pharmaceutical composition of claim 1 wherein it contains one or more suspension agents.

11. A pharmaceutical composition of claim 10, wherein the suspension agent or agents are bioadhesive silicic acid derivatives.

12. A pharmaceutical composition of claim 1 wherein the excipient is a fatty phase formed by semisynthetic glycerides.

13. A pharmaceutical composition of claim 11, wherein the semisynthetic glycerides are Witepsol® or Suppocire®.

14. A pharmaceutical composition of claim 1 which also contains one or more gelling agents.

15. A pharmaceutical composition of claim 14, wherein the gelling agent or agents are cellulose derivatives.

16. A pharmaceutical composition of claim 14, wherein the gelling agent is a carbomer.

17. A pharmaceutical composition of claim 16, wherein the gelling agent is polycarbophil in acid form or in salified form.

18. A pharmaceutical composition of claim 16 wherein the gelling agent is polycarbophil in the form of calcium salt.

19. A pharmaceutical composition of claim 1 having a sustained release of the active ingredients, spread over more than twenty four hours, wherein the excipient is a fatty material in which the oxybutynin hydrochloride is placed in suspension.

20. A pharmaceutical composition of claim 1, allowing T maxs of oxybutynin to be obtained between approximately two hours and approximately sixteen hours wherein the excipient or the vehicle is selected so that the speed of release is as long as possible.

21. A pharmaceutical composition of claim 1 wherein the excipient or the vehicle is selected so that the administration of oxybutynin takes place once, or optionally twice, per twenty four hours.

22. A method of treating urinary incontinency in humans comprising administrating to humans in need thereof an amount of a composition of claim 1 sufficient to treat urinary incontinency.
